(19) United States
(12) Patent Application Publication
(10) Pub. No.: US 2007/0124171 A1
(43) Pub. Date: May 31, 2007

(54) System and method for universal medication identification

(76) Inventor: William R. Benfield, Seattle, WA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/287,697

(22) Filed: Nov. 28, 2005

(51) Int. Cl. G06Q 10/00; A61K 49/00

(52) U.S. Cl. 705/2; 424/10.1

(57) ABSTRACT

A system or a method for identifying a pharmaceutical dosage form of a drug or medication is provided that improves safety in handling and administration of the drug or medication. A system for assigning a unique identification code to all dosage forms is provided to indicate specific classes of drugs, such as controlled substances.

### FIELD

The invention generally relates to a system or a method for identifying a pharmaceutical dosage form of a medication, drug, or biologic that improves safety in handling and administration of the medication, drug, or biologic. The invention further relates to a dosage form or a system to assign a unique identification code to each dosage form to indicate one of several specific classes of medication, drugs, or biologics, such as a controlled substance.

### BACKGROUND

A solid oral dosage form (SODF) is essentially a tablet or capsule. Most of the medication being consumed in the United States is fundamentally unsafe because it cannot be properly identified. Since 1995, it has been Federal law in the United States, that every SODF must have identification markings on the medication. However, the government does not dictate any particular form these identification codes must conform to. As a result, each manufacturer of medications has developed their own internal system for marking the SODF and complying with the statute. In the United States, there is currently no system that ties all of these codes together. Benfield, W.R., American Pharmacy, Vol. N832, No. 6, 44-47, 1992. Individuals, and more specifically, doctors, nurses, pharmacists, patients, police officers, and school officials need a reliable means to identify medications. It is important that all solid oral dosage forms have a reliable and identifiable coding system printed on the medications. This is especially true for all controlled substances or dangerous medications. A need exists in the art for an identification system that provides a consistent and identifiable code on all medication, generic or branded.

### SUMMARY

The present invention provides a system or a method for identifying all medications, for example, solid oral dosage forms (SODF), that substantially improves the safety in handling and administration of medications with a universal generic identification code. The invention further relates to a dosage form or a system or method to assign a unique identification code to every solid oral dosage form (SODF) e.g., tablets and capsules, based on the chemical contained in the medication and the strength of the medication. The system or method provides the unique identification code that indicates one of several specific classes of medication, drugs or biologics, such as a controlled substance. In many instances, this system or method has the ability to assign identification codes to medications in a manner such that simply looking at the code can give a person instant information about the medication. The unique identification code is generally envisioned for solid oral dosage forms, but can also be used for liquid dosage forms, and for dosage forms administered by means other than oral administration. The unique identification code is generally envisioned to identify all medications available in the United States, but is also applicable to medications available outside of the United States.

The present invention provides a system or a method for identifying a pharmaceutical dosage form of a medication, drug, or biologic that provides improves safety in handling and administration of the medication, drug, or biologic. The dosage form of the medication can be either generic or branded. The dosage form can be solid or liquid. The present system or method improves the ability of individuals, and more specifically, doctors, nurses, pharmacists, patients, police officers, or school officials to identify medications by providing a universal system that is readily accessible in a database, for example, electronically on the internet, or in a printed book, to identify all types of medications, drugs or biologics. For example, the present invention provides doctors, nurses, pharmacists, patients, police officers, or school officials with a reliable and understandable system of unique codes containing smart digits and therapeutic group codes to distinguish categories of solid oral dosage forms. The “smart digits” can distinguish categories, for example, controlled substances, time-release medication, or two or more active ingredients. The “therapeutic group codes” can distinguish, for example, medications, drugs or biologics, (prescription or over the counter), vitamins, homeopathic medication, herbal remedies, veterinary medication, and non-medications or medications not meant to be taken internally. Additional “smart digits” or “therapeutic group codes” that distinguish medicines, drugs, or biologics, or other compounds, are within the scope of this invention.

A system or method for identifying all pharmaceutical dosage form, e.g., all solid oral dosage forms, is provided comprising a database of unique identifiers of letters or numbers, or symbols, or a combination thereof, assigned to a medication, drug, or biologic to indicate medication type and dosage strength, and an imprint of the unique identifier on the dosage form. In one aspect of the system, the unique identifier is a combination of letters and numbers. In one embodiment, the unique identifier is a combination of from two to four letters and from two to four numbers. In a detailed embodiment, the unique identifier is a combination of three letters and three numbers. The unique identifier can further comprise a therapeutic group code and a smart digit letter or number indicating a property or class of the medication, drug or biologic. The medication, drug or biologic can be prescription or over the counter. The medication, drug or biologic can be generic or branded.

In one embodiment of the invention, the unique identifier is a combination of three letters and three numbers. The method or system is exemplified in detail below. A system or method for identifying all solid oral dosage forms is provided comprising a database of unique identifiers. In this embodiment of the system or method, the unique identifiers are always in the same format, specifically three letters followed by three numbers. There are no exceptions within the system making the three letters and three numbers of the system instantly identifiable by an individual, public official, or health care worker. There are over 17 million possible combinations of letters and numbers in this format. The unique identifiers are assigned to the medication, drug, or biologic indicates medication type and dosage quantity, and an imprint of the unique identifier is on the dosage form. The six digits, e.g., three letters and three numbers, can fit on the tablet or capsule and still be legible by an individual. The unique identifier incorporates “smart digits” and “therapeutic group codes”, which give the person some instant information by simple looking at the dosage form.

The system or method for identifying a pharmaceutical dosage form, e.g., a solid oral dosage form, can be referred to as a Universal Generic Identification Code (GIC). The system can be implemented throughout the United States but need not be limited to the United States, as it is 100% applicable to any country in the world. A solid oral dosage form (SODF) is essentially a tablet or capsule however, it also includes dosage forms such a troches, wafers and lozenges. It is Federal law in the United States, that every SODF must have identification markings on the medication. However, the government does not dictate any particular form these identification codes must conform to. As a result, each manufacturer of medications has developed their own internal system for marking the SODF and complying with the statute. In the United States, there is currently no system that ties all of these codes together. For example, it is no more useful than trying to identify the owner of an automobile by his license plate number. The information is available to a person in the Department of Motor Vehicles. But to the average man on the street, the license plate number is meaningless. With this number you do not receive any instant information by just looking at the number. You cannot even tell the gender of the owner from the license plate number. In exactly the same way, the numbers, letters and symbols appearing on a tablet or capsule are of little value in identifying a medication. The average person has no reasonable chance of identifying a medication once it is out of the original container. In fact, a study done in 1991 discovered that even the pharmacist, the drug expert, was generally unable to identify an unknown medication without calling the poison control center. The poison control centers report that they are pleased if they are able to correctly identify 80% of the drug identification calls in less than 15 minutes.

The system and method as presented herein is referred to as The Universal Generic Identification Code (GIC). It is based on a concept requiring a unique identifier on each type of drug in a dosage form, in particular, a unique identifier for each of the thousands of solid oral dosage forms, or other solid or liquid dosage forms, that have been approved for manufacture, distribution, and sale in the United States by the U.S. Food and Drug Administration, or by regulatory agencies in any foreign country. In one embodiment, the GIC can be a combination of three letters and three numbers to identify medications, drugs, and biologics sold or distributed in the United States. In a more general example, the GIC can be a combination of letters and numbers, or the GIC can further include symbols. The GIC can be all numbers or all letters, or a combination of numbers, symbols, and letters. The GIC comprising the unique identifiers can be collected in a database. These unique identifiers can be collected in a database, which is available on the internet in electronic format or in a print book. A person having access to this database can immediately identify any solid or liquid dosage form by referencing the GIC in the print or electronic database. In a further aspect, the electronic database will be accessible from an internet website for instant access by the interested person. The interested person can be a physician, nurse, pharmacist, patient, police officer, school official, or just an average citizen needing information.

A pharmaceutical dosage form includes, but is not limited to, medications and pharmaceutical compositions including drugs and biologics as defined by the U.S. Food and Drug Administration. A pharmaceutical dosage form further includes, but is not limited to, vitamins, homeopathic medication, veterinary medication, and non-medications or medications not meant to be taken internally The dosage forms can be available as either prescription medication or over-the-counter medication. The dosage forms can be solid or liquid. The dosage forms can be provided for oral, rectal, intravenous, intramuscular, subcutaneous, intraocular, intraaural, or intraperitoneal administration. In an exemplary embodiment, the pharmaceutical dosage form is a solid oral dosage form (SODF).

### BRIEF DESCRIPTION OF THE DRAWINGS

FIG. 1 shows examples of therapeutic group codes.

FIG. 2 shows examples of homeopathic medication codes.

FIG. 3 shows examples of herbal remedy codes.

FIG. 4 shows examples of veterinary medication codes.

FIG. 5 shows examples of non-medications in tablet or capsule form codes.

### DETAILED DESCRIPTION

In an embodiment of the invention, the system and methods referred to as the Universal Generic Identification Code utilizes six characters printed on a solid oral dosage form (SODF), comprising three letters and three numbers. In this embodiment, the six digits will always be in the same format of three letters followed by three numbers. Every manufacturer of a particular medication will place the same code on each SODF. For example, every manufacturer in the United States of Ibuprofen 400 mg will place the code IBU 400 on their product. It will make no difference if the tablet is round or square. The color of the medication will be of no consequence. The dosage form can be a tablet, capsule, or other solid or liquid dosage form. For this system or method, if it has the code IBU 400 printed on the tablet or capsule, it is Ibuprofen 400 mg. In most instances, there will be sufficient room on the tablet or capsule for the manufacturer to place their logo or number on the SODF.

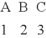

In this example, Universal Generic Identification Code (GIC) is 6-digits. It always consists of a three letters followed by three numbers. Every GIC in the database is in this format. If it is anything different, it is not a Universal Generic Identification Code (GIC) as devised for this particular example.

A further aspect of the Universal Generic Identification Code (GIC) is a unique process used to generate these code numbers. The system utilizes “Smart Digits” and “Therapeutic Group Codes” to arrive at a code capable of giving a person instant information by simply looking at the number. An exemplary process of assigning the code is described in further detail below. Other coding systems that provide smart digits and therapeutic group codes to identify important classes of compounds are considered within the aspects of this invention. Smart digits and therapeutic group codes identify classes of compounds including, but not limited to, controlled substances, time-release medication, medication with two or more active ingredients, vitamins, homeopathic medication, herbal remedies, veterinary medication, and non-medications or medications and chemicals in tablet or capsule form not meant to be taken internally.

The “smart digits” can distinguish categories such as controlled substances, time-release medication, or two or more active ingredients. The “therapeutic group codes” can distinguish medications (prescription or over the counter), vitamins, homeopathic medication, herbal remedies, veterinary medication, and non-medications or medications and chemicals in tablet or capsule form not meant to be taken internally. Additional “smart digits” or “therapeutic group codes” that distinguish medicines, drugs, or biologics, are within the scope of this invention.

### The Process Used to Generate Each GIC Code

Start with a piece of paper with six squares. Draw two rows with three squares.

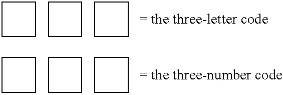

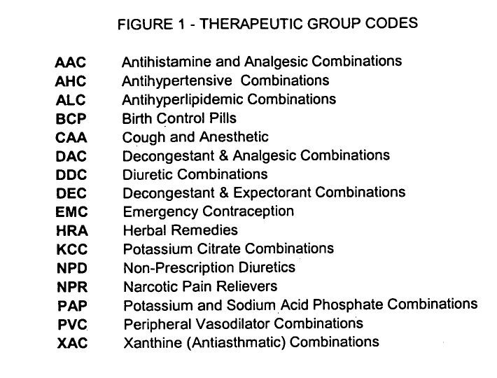

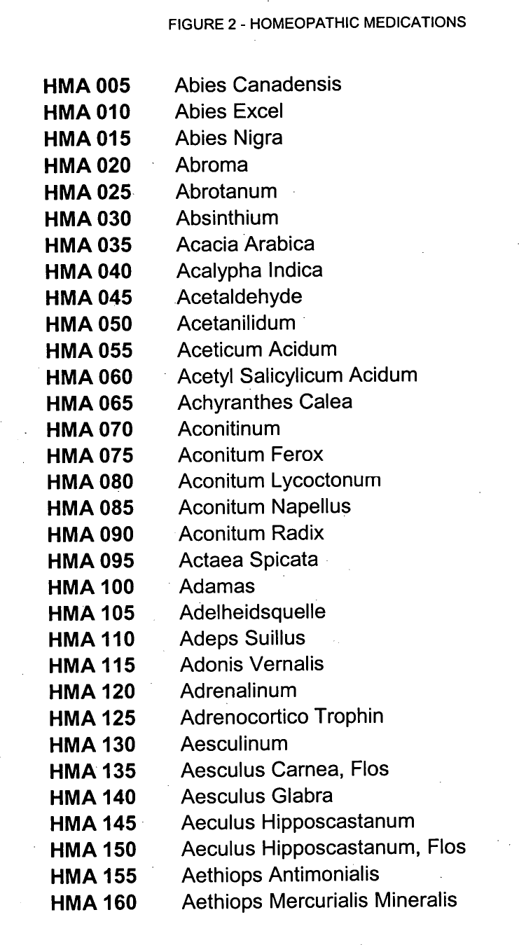

The first thing that must be done is to apply any of the applicable “Smart digits,” or “Therapeutic Group Codes.” The “Smart digits” include, but are not limited to, 9 (to indicate a controlled substance), 7 (to indicate a time-released substance), 4 (to indicate more than one active ingredient), and V (to indicate a vitamin). The “Therapeutic Group Codes” are three letters and indicate a medication with more than one active ingredient. Single active ingredients are identified by a three-letter code, and the three-letter code generally indicates the name of the active ingredient.
1.) Is the tablet or capsule a Controlled Substance?
   Yes =The first smart digit of the Three-Number Code will be 9. Write “9” in the first box of the Three-Number Code. Go to Step 2.
   No =Go to Step 2.
2.) Is the tablet or capsule time released? (Anything other than regular release qualifies as time released. Includes: Time-Released, Delayed Release, Enteric Coated, and all other variations.)
   Yes =The last digit of the Three-Number Code will be 7. Write “7” in the last box of the Three-Number Code. Go to Step 3.
3.) Does the tablet or capsule have more than ONE active ingredient?
   Yes =The last digit of the Three-Number Code will be 4. A “7” takes precedence over a “4”. If you have not already placed a “7” in the last box of the Three-Number Code, write “4” in the box.
      Also,
   Yes =The Three-Letter Code will come from the list of
      “Therapeutic Group Codes”. (Exceptions include vitamins, minerals, nutritional supplements, homeopathic medications, herbal remedies, veterinary medications and non-medications, which are dealt with separately. See Steps 4 through 8 below.) An exemplary list of the Therapeutic Group Codes is shown, for example, in FIG. 1. Refer to the Therapeutic Group Codes and select the appropriate code for the medication. Write the three letters in the Three-Letter Code boxes.
      Also,
   Yes =The first two digits of the Three-Number Code will NOT be derived from the strength of the medication, but instead will be assigned the next sequential number in the series starting with “00”. Refer to the current GIC listing arranged alphabetically by GIC Code to determine the last number assigned in this Therapeutic Group. Add one (1) to this two-digit number and write the new number in the first two boxes of the Three-Number Code, provided that the medication is not a vitamin, mineral, nutritional supplement, homeopathic medication, herbal remedy, veterinary medication and non-medication, which are dealt with separately. See Steps 4 through 8 below. The GIC code for this medication should now be complete. Go to Step 4.
   No =Go to Step 4.
4.) Is the tablet or capsule a vitamin? (This also includes minerals or nutritional supplements)
   Yes =The first letter of the Three-Letter Code will start with V. Anything starting with a “V” is a vitamin (including minerals and nutritional supplements.) Write “V” in the first box of the Three-Letter Code.
      The last two letter are a function of the vitamin, mineral or nutritional supplement being named. For example, vitamins generally referred by a letter, like vitamin C, would use that letter as the second letter of the Three-Letter Code. The final letter could be any letter still available.
      In the case of minerals, the international chemical symbols are the first choice for the last two letters. For example, FE for iron, CA for calcium, MG for magnesium, etc.
      For nutritional supplements, if available, the first letter of the two main ingredients in the product are generally used to determine the last two letters; however, there is no special approach. Go to Step 5.
   No =Go to Step 5.
5.) Is the tablet or capsule a Homeopathic medication?
   Yes =The Three-Letter Code will be HMA through HMZ (A total of 26 combinations all starting with HM.) There are thousands of Homeopathic medications. All of those starting with the letter “A” will have the Three-Letter Code HMA. Those starting with “B” will use the code HMB and so on down to HMZ.
      Strengths will NOT be used to assign the Three-Number Codes for Homeopathic medications. The numbered list of Homeopathic medications must be consulted. An exemplary list of Three-Number Codes for Homeopathic medications is shown, for example, in FIG. 2.

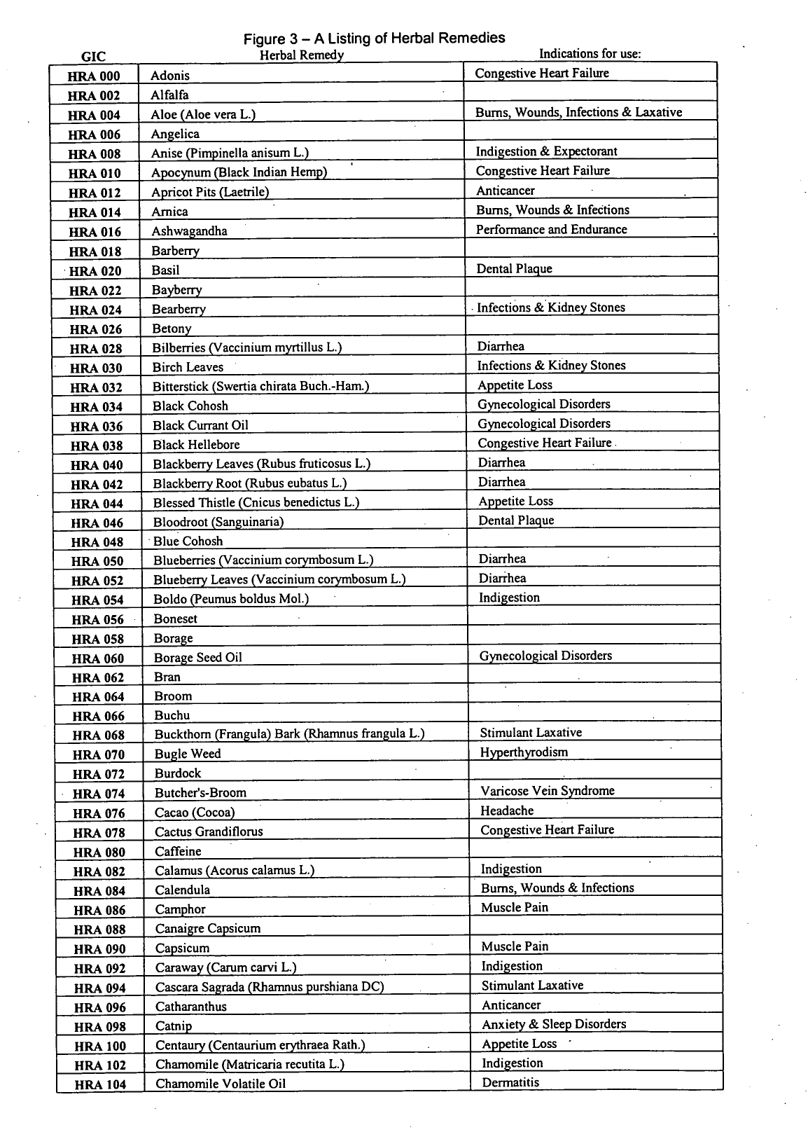

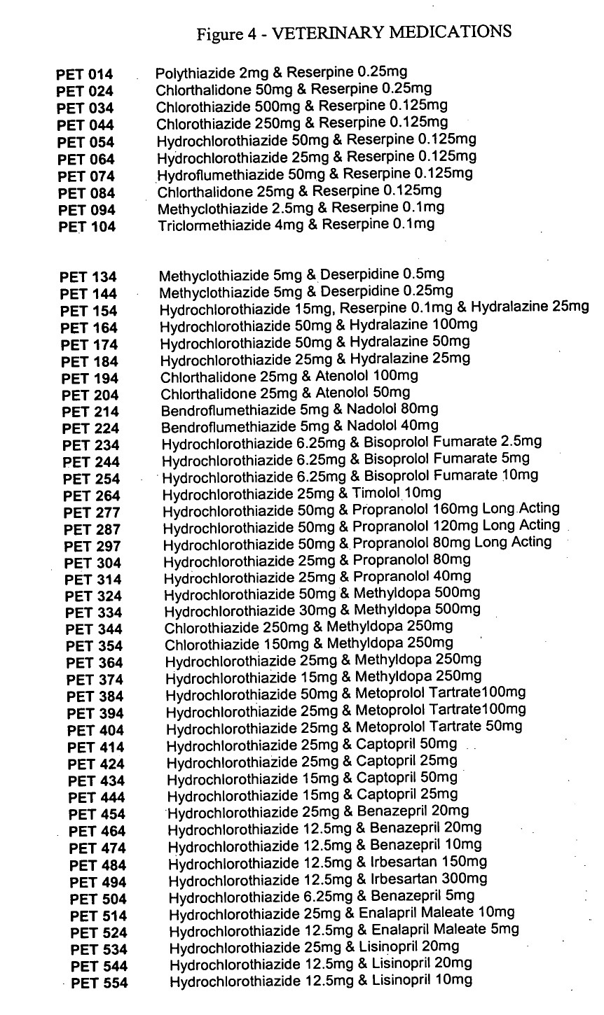

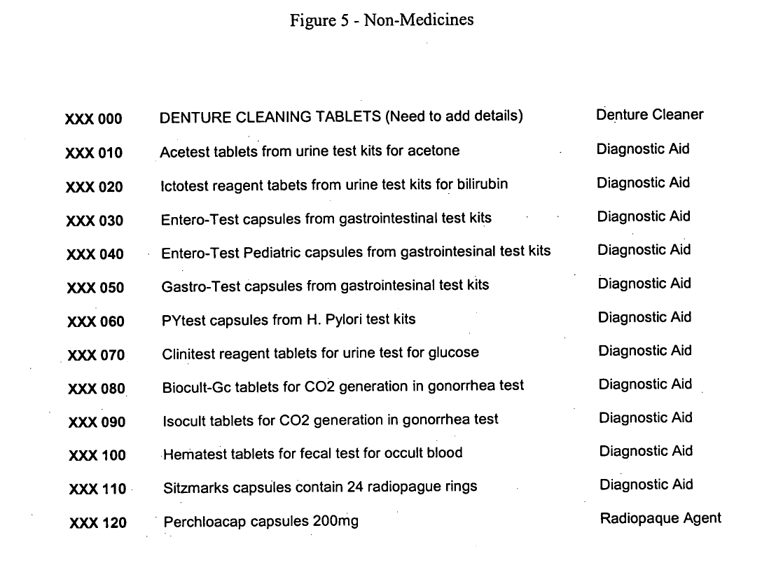

The three-digit number assigned to that entity or combination of entities is entered in the Three-Number Code boxes. The GIC code for this Homeopathic medication should now be complete. Go to Step 6.
No =Go to Step 6.
6.) Is the tablet or capsule a Herbal Remedy?
   Yes =The Three-Letter Code will be HRA through HRZ (a total of 26 combinations all starting with HR).
      There are hundreds of Herbal Remedies. All of those starting with the letter “A” will use the Three-Letter Code of HRA. Those starting with “B” will be assigned the code HRB and so on down to HRZ.
      Strengths will not be used to assign the Three-Number Codes for Herbal Remedies. A numbered list of Herbal Remedies must be consulted. An exemplary numbered list of Herbal Remedies is shown, for example, in FIG. 3. The three-digit number assigned to that entity or combination of entities and the strengths is entered in the Three-Number Code boxes. The GIC code for this Herbal Remedy should now be complete. Go to Step 7.
   No =Go to Step 7.
7.) Is the tablet or capsule a Veterinary medication?
   Yes =The Three-Letter Code will be PET. If more than a 1000 numbers are needed to code all the Veterinary medication; DOG or CAT will be used next, followed by COW, PIG and HEN. A numbered list of Veterinary medications must be consulted. An exemplary numbered list of Veterinary medications is shown, for example, in FIG. 4. The three-digit number assigned to that entity or combination of entities and the strengths is entered in the Three-Number Code boxes. The GIC code for this Veterinary medication should now be complete. Go to Step 8.
   No =Go to Step 8.
8.) Is the tablet or capsule a NON-medicine? For a variety of reasons, some chemicals are prepared and distributed in tablet form and they were never intend to be consumed by humans. In fact, consumption of these tablets or capsules could be FATAL. For example, there are some urine tests that require a reagent tablet to be placed in the test tube and be shaken.
   Yes =The Three-Letter Code will be XXX.
      A numbered list of these NON-medicines must be consulted. An exemplary numbered list of NON-medicines is shown, for example, in FIG. 5. The three-digit number assigned to that entity or combination of entities and the strengths is entered in the Three-Number Code boxes. The GIC code for this NON-medication should now be complete. Go to Step 9.
   No =Go to Step 9.
9.) If the tablet or capsule is not a vitamin, herbal remedy, homeopathic medication, veterinary medication or a NON-medication and it has only one active ingredient, then it must be assigned a full Three-Letter Code.

### How the Three-Letter Codes Are Assigned.

If the tablet or capsule has more than one active ingredient, go back to step 3 above.

Check to see what is available to be used. With 17 million possible combinations, there is a good chance whatever you want to assign to the medication won't be a problem.

Is there an existing abbreviation or three letter code for this medication? For example, Chlorpromazine is CPZ, Nitroglycerin is TNT. If such an abbreviation exists, it should be given serious consideration as the Three-Letter Code

On many occasions there are some very obvious three-letter combinations that are perfect for a medicine because it becomes a mnemonic devise and makes it much easier to remember. A few examples:
BCP 000 =The Inert tablets in Birth Control Pills
AWC 934 =Aspirin with Codeine #3's (30 mg)
TWC 934 =Tylenol with Codeine #3's (30 mg)
PMU 625=Premarin 0.625 mg (This medicine is made from Pregnant Mares Urine.)

Every effort must be made to NOT use Three-Letter Codes that are obscene, offensive or in poor taste. For example: SEX, DIE, TIT or BUM.

Every effort should be made to avoid Three-Letter Codes, which are well known corporations. For Example: ABC, NBC, CBS, UPS or IBM.

Whenever possible use the first letter of the generic name of the medication as the first letter of the Three-Letter Code. The exception to this is the letter “V”; it is used exclusively for vitamins, minerals and nutritional supplements. The letter “U” is very underutilized and the medication staring with the letter “V” (Verapamil, Valsartan, etc) should substitute the letter “U” where they would have normally used “V”. For example, the code for Valsartan is USR and the code for Verapamil is UER.
a.) Consider the first three letters of the generic name as the code.
b.) Consider the first letter of the first three syllables of the generic name.
10.) If all of the steps outlined above have not generated an acceptable Three-Letter Code, then randomly assign any unused Three-Letter Code to the medication.

### How the Three-Number Codes Are Assigned.

Try NOT to use a Three-Number Code that may be offensive or cause unwarranted concern for a patient: For example 013 (unlucky number), 666 (religious meaning) or 911 (Emergency phone number). Try to avoid using 711 (the convenience store).
11.) Has the medication already been assigned a starting “9” or an ending “7” or “4” ?
   Yes =for this tablet or capsule BOTH a starting “9” AND an ending “7” or “4” have already been assigned. (The most difficult scenario, because you only have the center number unassigned, which means you can only accommodate 10 items.)
   When the medication has already been assigned a “9” to start the code AND a “7” or “4” to end the Three-Number Code, the medications involved are arranged in ascending order with the lowest strength first. The lowest strength is assign the number “0”, the next strength is “1” and so on up to “9”. A time-released narcotic with 4 different strengths
   would have the following Three-Number Codes: 907, 917, 927 and 937. Likewise, a narcotic pain reliever mixed with aspirin, thus having more than one active ingredient would end with a “4”. If there were 5 strengths of this medication, the Three-Number Codes would be: 904, 914, 924, 934 and 944.
      It is common for medications to have both normal release and time-released dosage forms. If it is also a narcotic it can be a little confusing if you don't follow the rules. For example: Morphine Sulfate is available in the following strengths:
      Normal Release=10 mg, 15 mg and 30 mg, Timed Release=15 mg, 20 mg, 30 mg, 50 mg, 60 mg, 90 mg, 100 mg, 120 mg and 200 mg
      The Three-Letter Code for Morphine Sulfate is MOS.
   First, the normal release versions of the medication are assigned numbers, just like it was done in the last example. The different strengths are arranged in ascending order and the lowest strength is assigned “0” and so on. Because this medication is normal release, there is no “Smart Digit” ending the Three-Number Code. You are free to place any number, except “7” or “4” not currently assigned in the final position . In this example “0” will be used.
      The normal release SODF will have the following codes; 10 mg=MOS 900 15 mg=MOS 910 30 mg=MOS 920
      The timed release version of the morphine are arranged in ascending order with the lowest strength first. The lowest strength is assigned “0”, the next lowest strength is assigned “1” and so on up to “9”. The Three-Number Code becomes: 907, 917, 927, 937 . . . 987, 997.
      The timed release SODF will have the following codes: 15 mg=MOS 907 20 mg=MOS 917 30 mg=MOS 927 50 mg=MOS 937 60 mg=MOS 947 90 mg=MOS 957 100 mg=MOS 967 120 mg=MOS 977 200 mg=MOS 987
      If there are more than 10 items, the excess over 10 must be assigned a new Three-Letter Code. When this happens, it is best if the second Therapeutic Group Code is just the next letter in the alphabet. For example if the code is MOS 997, then the next medication should be MOT 907, MOT 917 etc.
   Yes =the medication has been assigned only a starting number “9” and does not also have an ending “7” or “4”.
      If the medication already has been assigned a “9” (Controlled Substance) to start the number, then the different strengths of the medication are arranged in ascending order and the lowest strength is assigned “00”, the next lowest strength is assigned “02” and so on, except no numbers ending with a 4 or 7 are used. So, the Three-Number Code becomes 9 plus the two digits: 901, 902, 903, 905, 906, 908, 909, 910 . . .995, 996, 998, 999. The numbers 904, 907, 914, 917 . . . 994 and 997 are not used to avoid any confusion with the time released medications (“7”) and the medicines that have more than one active ingredient (“4”).
   Yes=the medication has been assigned an ending number “7” or “4”.
      If the medication has already been assigned a “7” or “4” to end the Three-Number Code, then the medications involved are arranged in a list with the lowest strengths first. The lowest strength is assigned “01”, the next lowest strength is assigned “02” and so on up to “89”. No numbers in the nineties are used to help avoid any confusion that the medicine is a controlled substance. So, the Three-Number Code becomes the two digits plus the “7” or the “4”. For example: 017, 027, 037, 047, 057 . . . 867, 877, 887, 897. No numbers starting with a “9” are used. As a result, there would never be a number like 907, 917 or 927 unless it was a controlled substance. If more than 89 medications are assigned the same Three-Letter Code, the group will have to be split into two groups and assigned different Three-Letters Codes. Generally, some characteristic of the medications will easily allow the Therapeutic Group Code to be split in some logical way to form two or more new therapeutic group codes.
12.) Assign the Three-Number Code to the remaining medications. (Please note: These should all be single entity chemicals. If medications with more than one active ingredient remain, they should be re-evaluated and properly coded with a “4” at the end of the Three-Number Code. (Return to Step 3 above)
Whenever possible, the strength should be used for the Three-Number code. Arrange the medications in ascending order with the smallest strength first.
Are all of the strengths whole numbers? (Not numbers like 0.625, 12.5 and 0.4)
   Yes=Use the strength as the Three-Number Code. Some examples:
      1 mg=001, 010 or 100
      250 mg=250 or 025
      125μg=125
If the strength ends with a “7” or a “4” add one (1) to the number and use that as the Three-Number Code. For example:
   Levothyroxine 137μg=LEV 138
   Albuterol 4 mg=ALB 005
For strengths that have a decimal point, place the different strengths in ascending order and attempt to move the decimal point over one space to the right on all the medications. For example: Nitroglycerin is manufactured in 0.3 mg, 0.4 mg and 0.6 mg strengths. These numbers can be changed to 3, 4 and 6. However this presents a new problem because one of the numbers is a “4”. So, the numbers are changed to 30, 40 and 60. This would make the GIC for these medications: TNT 030, TNT 040 and TNT 060
Occasionally, the strengths can present a challenge. For example:
   Premarin 0.3 mg=PMU 003
   Premarin 0.625 mg=PMU 625
   Premarin 0.9 mg=PMU 009
   Premarin 1.25 mg=PMU 125
Slide the numbers back and forth until they all fit in the three-digit space.
For strengths with more than three digits, it is necessary to reduce it to only three digits.
For example:
   1000 mg=001 or 100
   1250 mg=125
   3375 mg =338 or any random number
When all of the easy and obvious Three-Number Codes have been assigned, simply assign three random numbers that have not already been used.
13.) The final step is to check that the newly assigned GIC does not already exist. In the case of a duplicate GIC, the newly assigned GIC must be changed. It is generally NOT AN OPTION to change the older GIC. If the older GIC has been published for any length of time, there could already be medicine with this GIC.

The disclosures of each patent, patent application and publication cited or described in this document are hereby incorporated herein by reference in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A system for identifying a pharmaceutical dosage form comprising:
a unique identifier assigned to the dosage form to indicate a medication name and a dosage strength,
a smart digit or a therapeutic group code, or a combination thereof, within the unique identifier to indicate a property or a class of the dosage form,
an imprint of the unique identifier on the dosage form, and
a database of the unique identifier assigned to the dosage form.

2. The system of claim 1, wherein the unique identifier comprises letters or numbers, or a combination thereof.

3. The system of claim 2, wherein the unique identifier is a combination of from two to four letters and from two to four numbers.

4. The system of claim 3, wherein the unique identifier is a combination of three letters and three numbers.

5. The system of claim 4, wherein the unique identifier is a combination of three letters followed by three numbers.

6. The system of claim 1 wherein the unique identifier is assigned to every pharmaceutical dosage form for sale in the United States.

7. The system of claim 1, wherein the smart digit indicates a controlled substance.

8. The system of claim 1, wherein the smart digit indicates a timed release medication.

9. The system of claim 1, wherein the smart digit indicates two or more active ingredients.

10. The system of claim 1, wherein the smart digit indicates a vitamin.

11. The system of claim 1, wherein the therapeutic group code indicates an herbal remedy.

12. The system of claim 1, wherein the therapeutic group code indicates a homeopathic medication.

13. The system of claim 1, wherein the therapeutic group code indicates a veterinary medication.

14. The system of claim 1, wherein the therapeutic group code indicates a non-medication.

15. The system of claim 1 wherein the database is in print form or electronic form.

16. A method for identifying a pharmaceutical dosage form comprising
assigning a unique identifier to the dosage form indicating a medication name and a dosage strength,
assigning a smart digit or a therapeutic group code, or a combination thereof, within the unique identifier to indicate a property or a class of the dosage form,
imprinting the unique identifier on the dosage form, developing a database of the unique identifier assigned to each dosage form.

17. The method of claim 16 wherein the unique identifier comprises letters or numbers, or a combination thereof.

18. The method of claim 16, wherein the unique identifier is a combination of from two to four letters and from two to four numbers.

19. The method of claim 18, wherein the unique identifier is a combination of three letters and three numbers.

20. The method of claim 19, wherein the unique identifier is a combination of three letters followed by three numbers.

21. The method of claim 16, further comprising assigning the unique identifier to every dosage form for sale in the United States.

22. The method of claim 16, wherein the smart digit indicates a controlled substance.

23. The method of claim 16, wherein the smart digit indicates a timed-release medication.

24. The method of claim 16, wherein the smart digit indicates two or more active ingredients.

25. The method of claim 16, wherein the smart digit indicates a vitamin.

26. The method of claim 16, wherein the therapeutic group code indicates an herbal remedy.

27. The method of claim 16, wherein the therapeutic group code indicates a homeopathic medication.

28. The method of claim 16, wherein the therapeutic group code indicates a veterinary medication.

29. The method of claim 16, wherein the therapeutic group code indicates a non-medication.

30. The method of claim 16 wherein the database is in print form or electronic form.

31. A pharmaceutical dosage form comprising a unique identifier imprinted on the dosage form to identify a medication name and a dosage strength, wherein the unique identifier comprises a smart digit or a therapeutic group code, or a combination thereof, to identify a property or a class of the dosage form.

32. The dosage form of claim 31 further comprising a database of the unique identifiers cross referenced to the medication name, the dosage strength, and the property or class.

33. The dosage form of claim 31 wherein the unique identifier is a combination of letters and numbers.

34. The dosage form of claim 33, wherein the unique identifier is a combination of from two to four letters and from two to four numbers.

35. The dosage form of claim 34, wherein the unique identifier is a combination of three letters and three numbers.

36. The dosage form of claim 35, wherein the unique identifier is a combination of three letters followed by three numbers.

37. The dosage form of claim 31 wherein the unique identifier is assigned to every dosage form for sale in the United States.

38. The dosage form of claim 31, wherein the smart digit indicates a controlled substance.

39. The dosage form of claim 31, wherein the smart digit indicates a timed release medication.

40. The dosage form of claim 31, wherein the smart digit indicates two or more active ingredients.

41. The dosage form of claim 31, wherein the smart digit indicates a vitamin.

42. The dosage form of claim 31, wherein the therapeutic group code indicates an herbal remedy.

43. The dosage form of claim 31, wherein the therapeutic group code indicates a homeopathic medication.

44. The dosage form of claim 31, wherein the therapeutic group code indicates a veterinary medication.

45. The dosage form of claim 31, wherein the therapeutic group code indicates a non-medication.

46. The dosage form of claim 31, wherein the database is in print form or electronic form.

47. The dosage form of claim 31, wherein the dosage form is solid or liquid.

48. The dosage form of claim 31, wherein the dosage form is a solid oral dosage form.

49. The dosage form of claim 48, wherein the dosage form is a tablet or capsule.

50. The dosage form of claim 31, wherein the dosage form is generic or branded.

51. The dosage form of claim 31, wherein the dosage form is provided for oral, rectal, intravenous, intramuscular, subcutaneous, intraocular, intraaural, or intraperitoneal administration.
